(19) United States
(12) Patent Application Publication
(10) Pub. No.: US 2016/0305082 A1
(43) Pub. Date: Oct. 20, 2016

(54) Stabilization Machine Connected Site

(71) Applicant: Caterpillar Paving Products Inc., Brooklyn Park, MN (US)

(72) Inventors: Brian Joe Schlenker, Shoreview, MN (US); Jason William Muir, Andover, MN (US)

(73) Assignee: Caterpillar Paving Products Inc., Brooklyn Park, MN (US)

(21) Appl. No.: 14/687,661

(22) Filed: Apr. 15, 2015

(51) Int. Cl. E02D 3/12; G01N 33/24; E02D 3/10; E02D 1/04; E02D 3/00; E02D 3/026

(52) U.S. Cl. CPC E02D 3/12; E02D 3/005; E02D 3/026; E02D 3/10; E02D 3/126; E02D 1/04; G01N 33/246

(57) ABSTRACT

A system and method are disclosed. The system may comprise a first stabilization machine and one or more other machines or supply trucks. The system may also include one or more site controllers. The first stabilization machine may include a first stabilization controller. The first stabilization controller may be configured to receive location data and metering information, and to determine and transmit one or more of the following to the appropriate other machine(s), supply truck(s) or site controller(s): resupply time, location/boundaries of the worked surface area, the quantity of passes over the worked surface area, the mixing depth and the moisture content. The other machine(s), supply truck(s) or site controller(s) may each be in communication with user interface(s) on which the transmitted information may be displayed.

### TECHNICAL FIELD

The present disclosure generally relates to soil stabilization machines and, more particularly, to rotary mixers utilized in soil stabilization applications, and the like.

### BACKGROUND

Soil stabilization is the process of mechanically or chemically improving the load-bearing capacity of soil or a ground surface. The process of soil stabilization may be required where roads are constructed for vehicular travel, as well as for building pads on which other construction may take place. In addition, soil stabilization may be useful in other applications, including surface mining, bio-remediation, agriculture and the building of high strength haul roads.

Stabilization machines, such as rotary mixers, typically include a frame quadrilaterally supported on traction units, an engine, an operator's station and a hood member under which a milling/mixing rotor is disposed, thereby forming an open bottom mixing chamber. A rotary mixer may be used as a soil stabilizer to cut, pulverize and mix native in-place soils with additive or alternatively additive and a fluid.

Some stabilization machines or rotary mixers may include an additive storage container to hold the dry additive and a fluid reservoir to hold fluid. Such stabilization machines further include an apparatus for spreading the dry additive and fluid directly onto the base material before the mixing rotor travels over the base material. However, as the built-in/mounted storage container and fluid reservoir each have limited volume capacity, numerous refilling operations of the container and fluid reservoir may be required during a single stabilization process. Alternatively, a dry additive storage truck and a fluid storage truck may directly supply the stabilization machine during operation with dry additive and fluid until the respective truck supply is depleted. Both such trucks have a much larger storage capacity than an additive storage container or fluid reservoir mounted on the frame of the stabilization machine.

Once the supply of the dry additive or fluid is depleted, a refill or new supply must be obtained from a supply truck. The supply truck must travel to the jobsite. A stabilization machine may be left waiting for the supply truck to arrive at the jobsite and locate the appropriate stabilization machine.

On jobsites, compactors are commonly used to compact to a desired density the surface area that has been worked by the stabilization machine. The number of passes of the stabilization machine over a given surface area, the mixing depth of the rotor of the stabilization machine and the moisture content of the resulting worked ground may impact the operation of the compactor to obtain the desired density.

On some jobsites and projects, multiple stabilization machines may work together to prepare adjacent or related portions of a surface area. It is often desirable to maintain consistency between the stabilization machines working different portions of a surface area with regard to the number of passes made, the mixing depth of the rotor and the moisture content of the resulting worked ground.

U.S. Pat. No. 7,489,993 issued Feb. 10, 2009 (the '993 Patent) discloses a vehicle fleet management information system that identifies location and direction of movement of each vehicle in a fleet and reports such information to a fleet manager. While beneficial, this system does not provide information to a supply truck on when the dry additive or fluid applied by a stabilization machine should be resupplied. A better system is needed.

### SUMMARY OF THE DISCLOSURE

In accordance with one aspect of the disclosure, a system is disclosed. The system may comprise a first stabilization machine and a supply truck. The first stabilization machine may include a first stabilization controller. The first stabilization controller may be configured to determine a resupply time for a dry additive or fluid that is applied by the first stabilization machine, and to transmit to a supply truck controller the resupply time and a current location of the first stabilization machine. The supply truck may be configured to transport the dry additive or the fluid. The supply truck may include a supply truck user interface and the supply truck controller. The supply truck controller may be in communication with the first stabilization controller and the supply truck user interface. The supply truck controller may be configured to display on the supply truck user interface the resupply time and the location.

In accordance with another aspect of the disclosure, a method of coordinating activities of a first rotary mixer in a system is disclosed. The system may include the first rotary mixer and a supply truck. The first rotary mixer may include a first stabilization controller. The supply truck may include a supply truck controller and a supply truck user interface. The supply truck controller may be in communication with the first stabilization controller and the supply truck user interface. The method may comprise: determining, by the first stabilization controller, a resupply time for a dry additive or a fluid that is applied by the first rotary mixer; transmitting, by the first stabilization controller, the resupply time and a current location of the first rotary mixer to the supply truck controller; and displaying the resupply time and the current location on the supply truck user interface.

In accordance with a further aspect of the disclosure, a system is disclosed. The system may comprise a first rotary mixer, a compactor and a second rotary mixer. The first rotary mixer may include a first stabilization controller. The first stabilization controller may be configured to determine for the first rotary mixer a resupply time for a dry additive or fluid that is applied by the first rotary mixer, a location of a worked surface area that has been worked by the first rotary mixer, a quantity of passes made in a portion of the worked surface area by the first rotary mixer, a mixing depth of the worked surface area and a moisture content of the worked surface area. The first stabilization controller may be further configured to transmit to a compactor controller and to a second stabilization controller the location of the worked surface area, the quantity of passes, the mixing depth, and the moisture content. The compactor may include a compactor user interface and the compactor controller. The compactor controller may be in communication with the compactor user interface and the first stabilization controller. The compactor controller may be configured to display on the compactor user interface the location of the worked surface area, the quantity of passes, the mixing depth and the moisture content. The second rotary mixer may include a stabilization user interface and the second stabilization controller. The second stabilization controller may be in communication with the stabilization user interface and the first stabilization controller. The second stabilization controller may be configured to display on the stabilization user interface the location of the worked surface area, the quantity of passes, the mixing depth and the moisture content.

### BRIEF DESCRIPTION OF THE DRAWINGS

FIG. 1 is a general schematic view of an exemplary embodiment of a system constructed in accordance with the teachings of this disclosure;

FIG. 2 is an exemplary stabilization machine, partly in section, showing the interior of a mixing chamber;

FIG. 3 is a simplified schematic representation of connections between several elements in accordance with the teachings of this disclosure;

FIG. 4 illustrates an exemplary combination of interconnected machines in accordance with the teachings of this disclosure;

FIG. 5 illustrates an alternative exemplary combination of interconnected machines in accordance with the teachings of this disclosure;

FIG. 6 is a flowchart illustrating exemplary steps of a method of coordinating the resupply of a stabilization machine by a supply truck in accordance with the teachings of this disclosure; and

FIG. 7 is a flowchart illustrating exemplary steps of a method of coordinating the activities of the first stabilization machine 102a and either a compactor 112 or a second stabilization machine 102b in accordance with the teachings of this disclosure.

### DETAILED DESCRIPTION

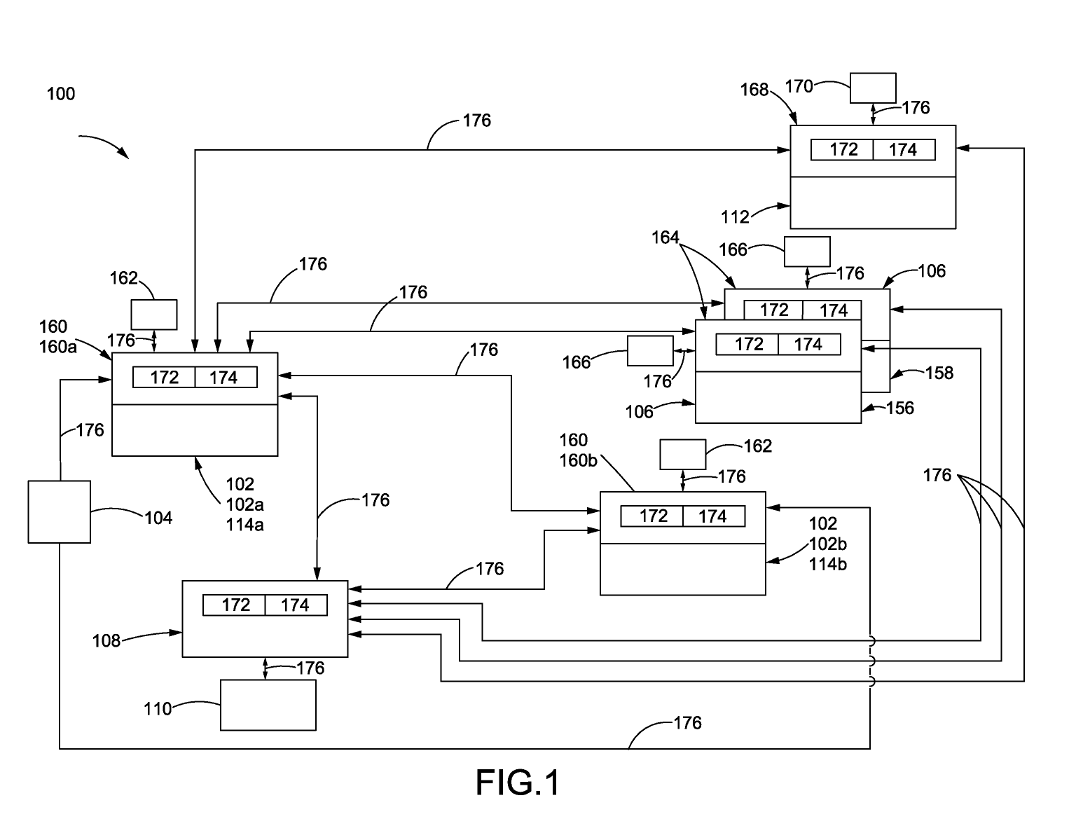

Referring now to the drawings and with specific reference to FIG. 1, there is shown a schematic diagram of an exemplary embodiment of a system in accordance with the present disclosure and generally referred to by reference numeral 100. The exemplary system 100 may comprise at least one stabilization machine 102, and at least one other machine. Each stabilization machine 102 may receive location data from a navigation system 104.

In one embodiment, the system 100 may comprise one or more stabilization machines 102 and one or more supply trucks 106 configured to transport dry additive or fluid. In another embodiment, the system 100 may comprise one or more stabilization machines 102 and one or more compactors 112. In yet another embodiment, the system 100 may comprise a first stabilization machine 102a, and a second stabilization machine 102b. Any of the above systems 100 may further include a site controller 108 and a site user interface 110. In other embodiments, the system 100 may include various combinations of the above. Other embodiments may include fewer or greater numbers of stabilization machines 102, fewer or greater numbers of supply trucks 106, fewer or greater numbers of compactors 112, more than one the site controller 108, and more than one site user interface 110. In some embodiments there may be no site controller 108 or site user interface 110.

One exemplary system 100 is shown in FIG. 1. The exemplary system 100 of FIG. 1 may comprise the first stabilization machine 102a, one or more supply trucks 106, the site controller 108, the site user interface 110, the compactor 112, and the second stabilization machine 102b.

In an embodiment, the stabilization machine 102 may be a rotary mixer 114. In an embodiment that includes multiple stabilization machines 102, the first stabilization machine 102a may be a first rotary mixer 114a and the second stabilization machine 102b may be a second rotary mixer 114b. Each stabilization machine 102 may further include a stabilization controller 160 and a stabilization user interface 162. While the description and drawings are made with reference to a stabilization machine 102 that is a rotary mixer 114, in other embodiments the stabilization machine(s) 102 may be any other type of machine used in road reclamation, soil stabilization, or surface pulverization.

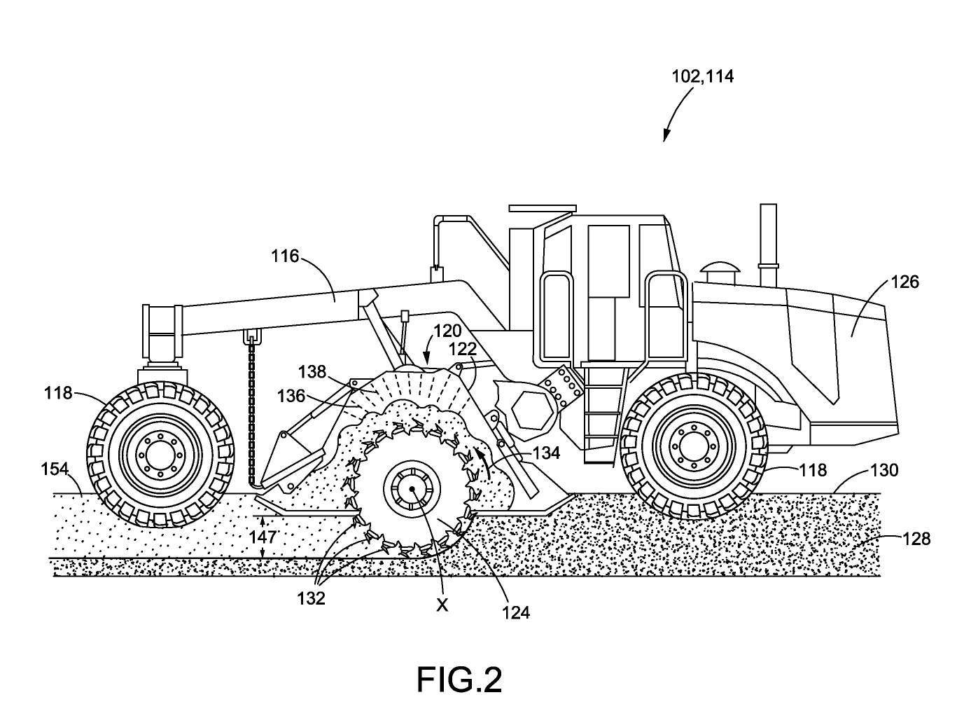

FIG. 2 illustrates one exemplary stabilization machine 102 that is a rotary mixer 114. The rotary mixer 114 may include a frame 116 connected to one or more wheel or traction units 118. Although traction units 118 are depicted as wheels, it is to be understood that other devices, such as but not limited to tracks or the like may also be employed. According to FIG. 2, the rotary mixer 114 may include an open bottom mixing chamber 120 positioned between the traction units 118. The mixing chamber 120 may generally extend the width of the rotary mixer 114. The mixing chamber 120 may be partially defined by a hood 122 and may include a ground engaging rotor 124 partially housed therein. The rotor 124 may rotate about a generally horizontal axis X. An engine 126 (or other power source) may be configured to electrically, mechanically, hydraulically and/or pneumatically power the fraction units 118 and the rotor 124.

The rotor 124 may include components rotationally driven by the engine 126 to pulverize a base material 128 of a ground surface 130 on which the rotary mixer 114 is operating. Ground surface 130 as used herein may include any base material 128 such as soil, dirt, gravel, sand, stones, concrete, pavement and the like. The rotor 124 may further include a plurality of cutting tools 132 spaced apart and pointed in a direction of rotation (indicated by an arrow 134) of the rotor 124, such that a tip end of each cutting tool 132 is driven into the base material 128 by the rotation of the rotor 124. As the rotary mixer 114 advances along the ground surface 130 to be stabilized, the rotor 124 and cutting tools 132 penetrate the ground surface 130 and lift the base material 128 from the ground surface 130, causing the base material 128 to move upwards into the mixing chamber 120, as indicated in FIG. 2.

Allocations of dry additive 136 and fluid 138 may be introduced into the mixing chamber 120 where they collide with the upwardly moving base material 128. In this manner, the base material 128 is cut and pulverized within the mixing chamber 120, as well as immediately mixed with the dry additive 136 and fluid 138.

Dry additives 136 for soil stabilization may include fly ash, lime and/or cement, or the like. Fluids 138 may be water, oil, or like liquids. The addition of fluid 138 into the mixing chamber 120 may aid in the mixing process by wetting the pulverized base material 128 and the dry additive 136. The hood 122 of the mixing chamber 120 may provide a support for systems such as spray bars, nozzles or other fixtures by which dry additives 136 and fluid 138 may be delivered to the mixing chamber 120 and mixed with the base material 128. For example, spray bars supported on the hood 122 and extending the width of the mixing chamber 120 may deliver dry additives 136 and fluid 138 to the interior of the mixing chamber 120. Alternatively or in addition, a series of spaced nozzles or tubes may be disposed in the hood 122 to deliver dry additives 136 and fluid 138 to the interior of the mixing chamber 120.

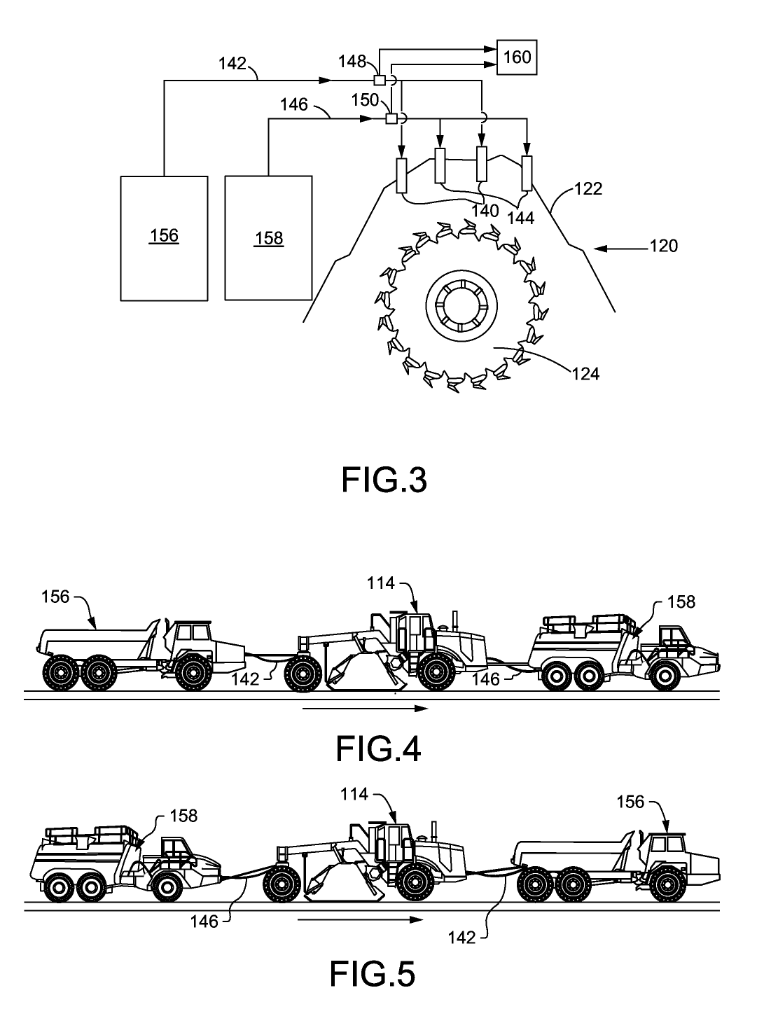

As illustrated in FIG. 3, a dry additive delivery system 140 employed to deliver dry additive 136 to the mixing chamber 120 may be coupled to a dry additive supply line 142. Likewise, a fluid delivery system 144 employed to deliver fluid 138, for example water, oil or the like, to the mixing chamber 120 may be coupled to a fluid supply line 146. The dry additive delivery system 140 and the fluid delivery system 144, as described above, may be spaced throughout the hood 122 of the mixing chamber 120 in any pattern that allows efficient delivery of dry additives 136 and fluid 138 across the width of the mixing chamber 120 to the subject base material 128 within the mixing chamber 120.

Several factors may influence the amount of dry additive 136 or fluid 138 to be delivered to the mixing chamber 120 and mixed with the base material 128, including: the initial characteristics of the ground surface 130 or the base material 128, the load-bearing capacity needed, the speed at which the rotary mixer 114 advances, the speed at which the rotor 124 rotates and the depth at which the rotor 124 engages the ground surface 130 (the “mixing depth 147”) (see FIG. 2). In order to consistently control the amounts of dry additive 136 and fluid 138 delivered to the mixing chamber 120, metering devices 148, 150 may be coupled with the dry additive supply line 142, and fluid supply line 146, or with the dry additive delivery system 140 and fluid delivery system 144. Specifically, the dry additive delivery system 140 or the dry additive supply line 142 may have a metering device 148 that is the same as or different from a metering device 150 for the fluid delivery system 144 or the fluid supply line 146. Alternatively, the metering devices 148, 150 and delivery systems (dry additive delivery system 140, fluid delivery system 144) may essentially be the same component, for example, a nozzle in the hood 122 of the mixing chamber 120, the nozzle including controllable valves for metering the amounts of dry additive or fluid delivered to the mixing chamber 120.

The metering devices 148, 150 for controlling/measuring the volume and rate of delivery of dry additive 136 and/or fluid 138 in the disclosed system 100 may include, but are not limited to, inlets, outlets, tubes, pumps, one or more rotary feeders, pressurized components, expandable components and/or one or more valves. Additionally, the metering devices 148, 150 contemplated herein may be automatically computer controlled based on the forward movement of the rotary mixer 114, on the rotation of the rotor 124 or on any other parameter such as the amount or gradation of the base material 128 estimated to exist in the mixing chamber 120 at any particular moment during operation. Alternatively, the metering devices 148, 150 may be controlled by an operator.

The supply truck 106 may be a dry additive storage truck 156 or a fluid storage truck 158. As noted earlier, embodiments of the system 100 may include a plurality of supply trucks 106. As such, embodiments of the system 100 may include a plurality of dry additive storage trucks 156 and a plurality of fluid storage trucks 158. The supply truck 106, for example the dry additive storage truck 156 or the fluid storage truck 158, may further include a supply truck controller 164 and a supply truck user interface 166. The fluid storage truck 158 may be a water storage truck, an oil storage struck, or the like. While the dry additive and fluid storage trucks 156, 158 of the present disclosure may be generally self-propelled and include a cab for a driver, dry additive and fluid storage trucks 156, 158 as disclosed herein also encompass non-self-propelled apparatus, for example, a truck chassis or trailer having a dry additive or fluid storage tank disposed thereon.

As noted previously, the resupply of dry additive 136 and fluid 138 to the rotary mixer 114 may occur after a storage container or fluid reservoir on the rotary mixer 114 is empty. Alternatively, in a scenario where a dry additive storage truck 156 and a fluid storage truck 158 may be connected to the rotary mixer 114 for continuously supplying the rotary mixer 114 (during operation) with dry additive 136 and fluid 138, resupply may occur when the dry additive storage truck 156 or fluid storage truck 158 supply is depleted or the content volume falls below a threshold.

As illustrated in FIG. 3, the dry additive supply line 142 couples the dry additive storage truck 156 to the dry additive delivery system 140. Likewise, the fluid supply line 146 couples the fluid storage truck 158 to the fluid delivery system 144. The dry additive and fluid supply lines 142, 146 may be similar or different in their construction and attachment to their respective storage trucks 156, 158; however, each functions as a conduit for delivery of dry additive or fluid to delivery systems 140, 144 and ultimately to the mixing chamber 120 of the rotary mixer 114. The dry additive and fluid supply lines 142, 146 may include a flexible hose, tubing or any such conduit capable of extending between and transferring contents between the storage trucks (dry additive storage truck 156, fluid storage truck 158) and the rotary mixer 114.

Fluid storage trucks 158 (for example, water storage trucks or oil storage trucks) are known in the art and may include various combinations of actuators, pumps and valves for delivering fluid 138.

Regarding dry additive 136 delivery from the dry additive storage truck 156 into the dry additive supply line 142 and ultimately to the rotary mixer 114, dry additives 136 may be fluidized and distributed using methods similar those of fluid distribution systems known in the art. Dry additive 136 delivery from the dry additive storage truck 156 and through dry additive supply line 142 may involve pressure manipulation through the use of air compressors, fans, suction fans or pump mechanisms. Blower/impeller systems having one or more blowers may also be employed in the presently disclosed system 100 and methods. In some embodiments, a metered amount of dry additive may be blown with a fan or compressed air system under the hood 122 and directly into the mixing chamber 120 after passing through the metering device 148 proximate to the mixing chamber 120. As with the metering of dry additive 136 and fluid 138 into the mixing chamber 120, delivery of the dry additive 136 and fluid 138 from the dry additive storage truck 156 and the fluid storage truck 158 to the dry additive supply line 142 and the fluid supply line 146, and ultimately to the mixing chamber 120, may be automatically computer controlled or controlled by an operator such that continuous delivery at a controlled rate and volume is maintained.

FIGS. 4-5 illustrates various embodiments of the rotary mixer 114 connected to the dry additive storage truck 156 and to the fluid storage truck 158. In some embodiments, the rotary mixer 114, the dry additive storage truck 156, and the fluid storage truck 158 may travel or advance at the same speed and over the same ground surface 130 during the soil stabilization process.

FIGS. 4-5 also illustrate the dry additive supply line 142 and the fluid supply line 146 that supply dry additive 136 and fluid 138, respectively, to the rotary mixer 114. As described above, the dry additive supply line 142 and the fluid supply line 146 may be any type of conduit known in the art capable of delivering dry additive 136 and fluid 138 to the rotary mixer 114.

The compactor 112 (FIG. 1) may include a body, a prime mover, such as an engine, a roller mounted on the body, a compactor controller 168, and a compactor user interface 170. The engine is part of a drive system that propels the compactor 112.

The navigation system 104 may be a Global Positioning System (GPS), a Global Navigation Satellite System (GNSS), or the like, as is known in the art for determining the location of a machine, such as the stabilization machine 102, the supply truck 106, and/or the compactor 112. The navigation system 104 may be configured to provide location data to the stabilization controller 160 for the stabilization machine 102. The location data may include the current location of the stabilization machine 102, and previous locations of the stabilization machine 102. Previous locations of the stabilization machine 102 may be utilized by the stabilization controller 160 to determine the location of the surface area worked by the stabilization machine 102 (the “worked surface area 154”) (FIG. 2) and the number of passes made over the worked surface area 154.

The site controller 108 (FIG. 1) and the site user interface 110 are typically disposed remote from a machine and are generally used by a site supervisor, coordinator, dispatcher or the like to monitor and coordinate the activity of the stabilization machine 102, the supply trucks 106, the compactor 112 and other machines on the site.

The site controller 108, the stabilization controller(s) 160, the supply truck controller(s) 164, and the compactor controller 168 may each include a processor 172 and a memory component 174. Each stabilization controller 160 may be in operable communication via communication links 176 with one or more supply truck controllers 164, one or more compactor controllers 168, one or more site controllers 108 and other stabilization controllers 160 (on other stabilization machines 102). The site controller 108 may be in operable communication with each stabilization controller 160, each supply truck controller 164, and each compactor controller 168.

The stabilization controller 160 may be in operable communication via communication links 176 with the stabilization user interface 162. The supply truck controller 164 may be in operable communication via communication links 176 with the supply truck user interface 166. The compactor controller 168 may be in operable communication via communication links 176 with the compactor user interface 170. The site controller 108 may be in operable communication via communication links 176 with the site user interface 110. Each user interface 110, 162, 166, 170 may be configured to be an input/output device, and may include a display screen.

The communication link 176 may be hardware and/or software that enables the transmission and receipt of data messages through a data link or a wireless communication link. The wireless communication link may include, for example, satellite, radio (voice and/or data), cellular, infrared, Ethernet, and the like.

The processor 172 may be a microprocessor or other processor as known in the art. The processor 172 may execute instructions and generate control signals for determining the time at which a new supply of dry additive 136 or fluid 138 will be needed by the stabilization machine 102, the location and/or boundaries of the surface area worked by the stabilization machine 102, the quantity of passes in such surface area, the mixing depth and the moisture content. Such instructions may be read into or incorporated into a computer readable medium, such as the memory component 174 or provided external to the processor 172. In alternative embodiments, hard wired circuitry may be used in place of, or in combination with, software instructions to implement a control method.

The term “computer readable medium” as used herein refers to any non-transitory medium or combination of media that participates in providing instructions to the processor 172 for execution. Such a medium may comprise all computer readable media except for a transitory, propagating signal. Forms of computer-readable media may include any medium from which a computer processor 172 can read.

The each controller 108, 160, 164, 168 is not limited to one processor 172 and memory component 174. Each controller 108, 160, 164, 168 may be several processors 172 and memory components 174.

Each stabilization controller 160 may be disposed on a stabilization machine 102, for example on a rotary mixer 114. The stabilization controller 160 may be configured to receive location data from the navigation system 104. The location data received may include the current location of the stabilization machine 102 (in this case the rotary mixer 114), and previous locations of the stabilization machine 102 (rotary mixer 114). The stabilization controller 160 may be configured to determine, from the location data, the location of the worked surface area 154), the boundaries of the worked surface area 154, and the number of passes made by the stabilization machine 102 over the worked surface area 154 or portions of the worked surface area 154.

The stabilization controller 160 may be further configured to receive metering information from the metering devices 148, 150. The metering information may include the volume delivered and the rate of delivery of dry additive 136 and/or fluid 138 by the stabilization machine 102. Based on this metering information, the stabilization controller 160 may, in some embodiments, calculate the amount of fluid 138 or dry additive 136 remaining in on-board storage container, on-board fluid reservoir, or in the respective supply truck 106. The stabilization controller 160 may be configured to calculate the time (the “resupply time”) at which a new supply of dry additive 136 or fluid 138 will be needed by the rotary mixer 114 given the volume delivered (applied by the stabilization machine 102) and the rate of delivery. In some embodiments, the stabilization controller 160 may calculate the remaining time period (for example, minutes, seconds, etc.) until a new supply of dry additive 136 or fluid 138 will be needed based on the current rate of delivery of the respective dry additive 136 or fluid 138. In addition, the stabilization controller 160 may also be configured to determine the mixing depth 147 and the moisture content of the base material 128 of the worked surface area 154.

The stabilization controller 160 may be configured to transmit to one or more supply truck controllers 164 and/or one or more site controllers 108 the current location of the stabilization machine 102 (in this case, a rotary mixer 114), and the resupply time or the remaining time period until resupply is desired.

As noted earlier, in some scenarios there may be coordination between multiple stabilization machines 102 (for example a first rotary mixer 114a that includes a first stabilization controller 160a and a second rotary mixer 114b that includes a second stabilization controller 160b). The first stabilization controller 160a may be configured to transmit to at least one compactor controller 168, a second stabilization controller 160b (and, in some embodiments, other additional stabilization controllers 160) and/or one or more site controllers 108 the current location of the first rotary mixer 114a, the location of the worked surface area 154 worked by the first rotary mixer 114a, the boundaries of such worked surface area 154, and the number of passes made by the first rotary mixer 114a over the worked surface area 154 or portions of the worked surface area 154. In an embodiment, the first stabilization controller 160a may be further configured to transmit to at least one compactor controller 168, and one or more site controllers 108 and/or the second stabilization controller 160b (and, in some embodiments, additional stabilization controllers 160) the mixing depth 147 and/or the moisture content. The second stabilization controller 160b is configured to receive the transmitted information discussed above. Similarly, the first stabilization controller 160a is configured to receive mixing depth 147 and/or moisture content from other stabilization controllers 160 as well as the current location of the other stabilization machines 102, the location and boundaries of their worked surface areas 154 (the respective surface areas worked by each of the other stabilization machines 102), and the number of passes made by the other stabilization machines 102 over their respective worked surface areas 154 or portions of their respective worked surface areas 154.

The site controller 108 may be configured to receive the current location of each rotary mixer 114 and to display on the site user interface 110 the resupply time or the remaining time period until resupply is desired, the location of the worked surface area 154 worked by each rotary mixer 114, the boundaries of the worked surface area 154, and the number of passes made by each rotary mixer 114 over its worked surface area 154 or portions of its worked surface area 154, the mixing depth 147 and/or the moisture content.

The supply truck controller 164 may be configured to receive from one or more stabilization controllers 160 and to display on the supply truck user interface 166 the current location of one or more rotary mixers 114, the resupply time or the remaining time period until resupply is desired

The compactor controller 168 may be configured to receive from one or more stabilization controllers 160 and to display on the compactor user interface 170 the current location of the rotary mixer(s) 114, the location of the worked surface area 154 worked by each rotary mixer 114, the boundaries of each rotary mixer's 114 worked surface area 154, and the number of passes made by such rotary mixer 114 over the worked surface area 154 or portions of the worked surface area 154, the mixing depth 147 and the moisture content.

The second stabilization controller 160b may be configured to receive from the first stabilization controller 160a and to display on the second stabilization user interface 162b the current location of the first rotary mixer 114a, the location of the worked surface area 154 worked by the first rotary mixer 114a, the boundaries of the worked surface area 154, and the number of passes made by the first rotary mixer 114a over the worked surface area 154 or portions of the worked surface area 154, the mixing depth and the moisture content. In embodiments that include additional stabilization machines 102, the second stabilization controller 160b may be configured to receive and to display on the second stabilization user interface 162b similar information for each of the additional stabilization machines 102.

Also disclosed is method of coordinating activities of a first rotary mixer 114a in a system 100. The system 100 may include the first rotary mixer 114a and one or more supply truck(s) 106. In some embodiments, the system 100 may include one or more compactor(s) 112 and/or one or more additional rotary mixer(s) 114 (for example, a second rotary mixer 114b). The first rotary mixer 114a may include a first stabilization controller 160a. Each supply truck 106 may include a supply truck controller 164 and a supply truck user interface 166. Each compactor 112 may include a compactor controller 168 and the second rotary mixer 114b may include a second stabilization controller 160b. Each supply truck controller 164 is in communication with the first stabilization controller 160a and the respective supply truck user interface 166. The method may comprise determining, by the first stabilization controller 160a, a resupply time for a dry additive 136 or a fluid 138 that is applied by the first rotary mixer 114a; transmitting, by the first stabilization controller 160a, the resupply time and a current location of the first rotary mixer 114b to one or more supply truck controller(s) 164; and displaying the resupply time and the current location on one or more supply truck user interface(s) 166. The method may further comprise determining, by the first stabilization controller 160, a location of a worked surface area 154 that has been worked by the first rotary mixer 114a. In some embodiments, the method may further comprise transmitting to one or more compactor controller(s) 168 the location of the worked surface area 154. The method may further comprise determining, by the first stabilization controller 160, a quantity of passes made by the first rotary mixer 114a in a portion of the worked surface area 154 and transmitting the quantity to the compactor controller 168. The method may further comprise determining, by the first stabilization controller 160, a mixing depth or a moisture content of the worked surface area 154, and transmitting the mixing depth 147 or the moisture content to the compactor controller 168. In other embodiments, the method may further comprise transmitting to a second stabilization controller 160 the location of the worked surface area 154. The method may further include determining, by the first stabilization controller 160, a quantity of passes made by the first rotary mixer 114a in a portion of the worked surface area 154 and transmitting the quantity to the second stabilization controller 160b. The method may further include determining, by the first stabilization controller 160a, a moisture content or mixing depth 147 of the worked surface area 154 and transmitting the moisture content or mixing depth 147 to the second stabilization controller 160b. In some embodiments, the method may further comprise transmitting, by the first stabilization controller 160a, to one or more site controller(s) 108 the resupply time and the current location, and displaying on site user interface(s) 110 the resupply time and the current location, wherein each site controller 108 and site user interface 110 are remote from the first rotary mixer 114a and each supply truck 106.

### INDUSTRIAL APPLICABILITY

In operation, the present disclosure can find application in any number of different industries, such as but not limited to machines for stabilizing soil prior to road construction or the like. Indeed, soil stabilization of a ground surface is oftentimes required before any road or building construction on the ground surface may proceed. The present disclosure may improve upon existing soil stabilization processes by minimizing stoppages in work caused by waiting for resupply of dry additives or fluid. The present disclosure may further improve work site efficiency identifying to coordinating machines the surface areas that have been worked by the stabilization machine 102.

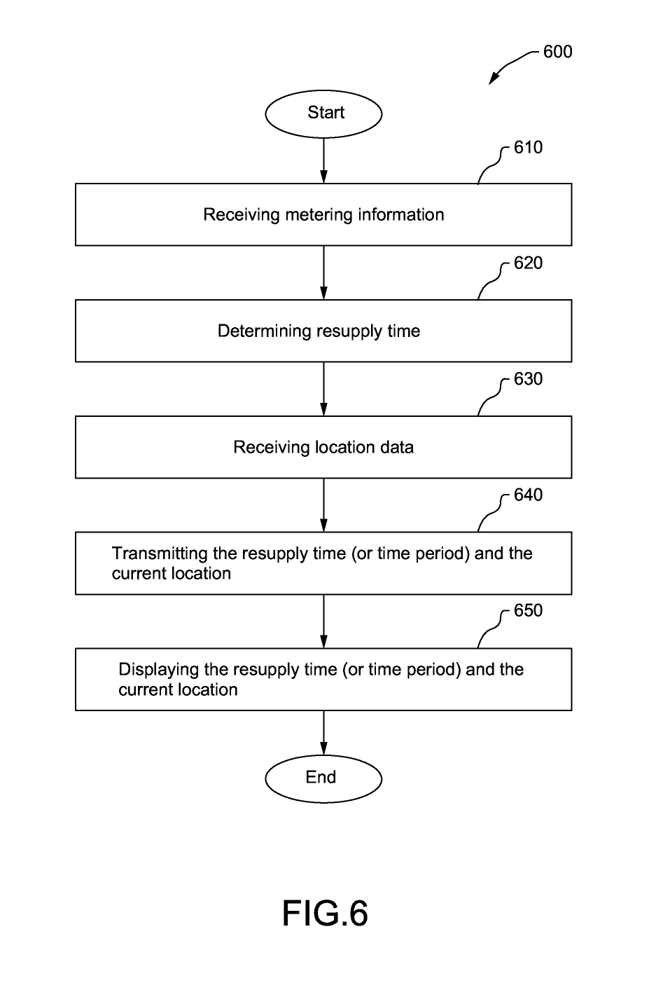

Referring now to FIG. 6, an exemplary flowchart is illustrated showing sample steps which may be followed to coordinate the resupply of the stabilization machine 102, for example a rotary mixer 114, by the supply truck 106. The method 600 may be practiced with more or less than the number of steps shown and is not limited to the order shown.

Block 610 of the method 600 disclosed herein includes receiving, by the stabilization controller 160, metering information from the metering devices 148, 150. The metering information may include the volume delivered and the rate of delivery of dry additive 136 and/or fluid 138 by the stabilization machine 102 (rotary mixer 114).

Block 620 includes determining the resupply time. Based on the metering information and the initial volume contained in the on-board container and/or fluid reservoir, or contained in the connected supply truck 106, the stabilization controller 160 may calculate the amount of dry additive 136 or fluid 138 remaining (in the on-board container or fluid reservoir or in the respective connected supply truck 106). The stabilization controller 160 may be further configured to calculate the time at which a new supply of dry additive 136 or fluid 138 will be needed by the rotary mixer 114 given the volume delivered and the rate of delivery. In some embodiments, the stabilization controller 160 may calculate the remaining time period until a new supply of dry additive 136 or fluid 138 will be needed based on the volume delivered and the rate of delivery of the respective dry additive 136 or fluid 138.

Block 630 of the method 600 includes receiving, by the stabilization controller 160 from the navigation system 104, location data for the stabilization machine 102 (in this case, the rotary mixer 114). The location data may include, but is not limited to, the current location of the stabilization machine 102.

Block 640 of the method 600 includes transmitting, by the stabilization controller 160 to one or more supply truck controllers 164 (on, for example, one or more dry additive storage trucks 156 or one or more fluid storage trucks 158), the resupply time (and/or time period) and the current location of the stabilization machine 102.

Block 650 includes displaying, on one or more supply truck user interfaces 166, the resupply time (and/or time period) and the current location of the stabilization machine 102. Such information is received by the supply truck user interface 166 from the supply truck controller 164.

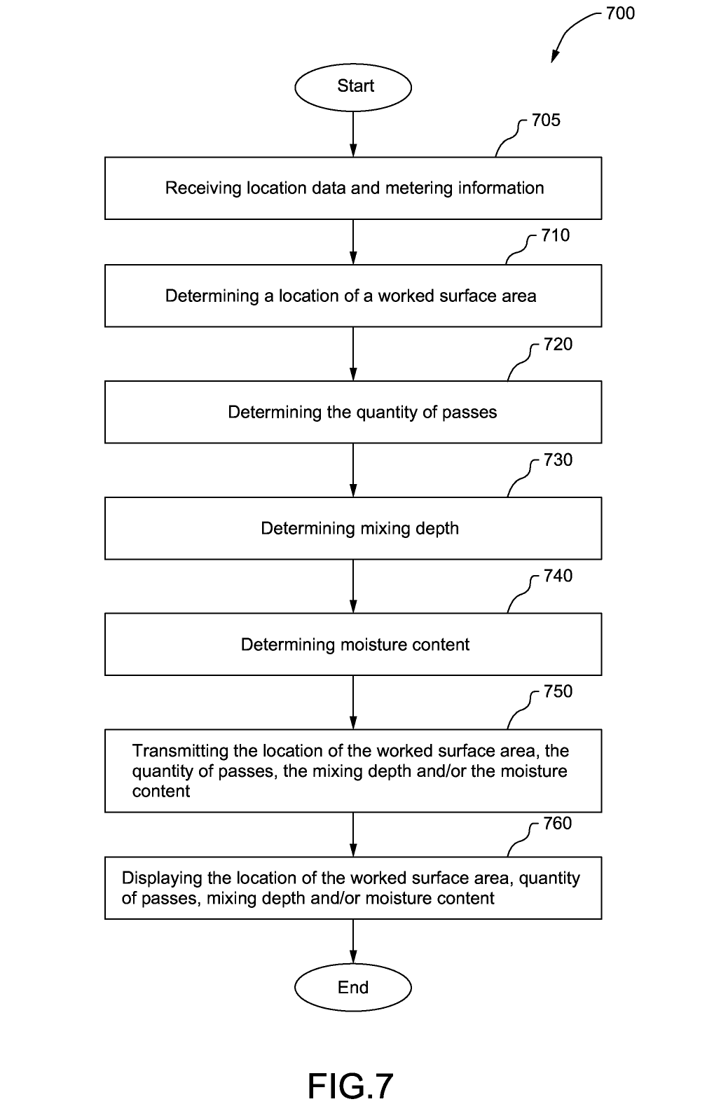

Referring now to FIG. 7, an exemplary flowchart is illustrated showing sample steps which may be followed in a method 700 to coordinate the activities of the first stabilization machine 102a, for example the first rotary mixer 114a, and either one or more compactors 112 or one or more additional stabilization machines 102 (for example, a second stabilization machine 102b such as a second rotary mixer 114b). The first stabilization machine 102a includes a first stabilization controller 160a. In an embodiment in which the system 100 includes a second stabilization machine 102b, the second stabilization machine 102b includes a second stabilization controller 160b. The method may be practiced with more or less than the number of steps shown and is not limited to the order shown.

Block 705 of the method 700 disclosed herein includes receiving, by the first stabilization controller 160a from the navigation system 104, location data for the first stabilization machine 102a (for example, location data for a first rotary mixer 114) and receiving metering information from the metering devices 148, 150. The location data may include, but is not limited to, the current location of the stabilization machine 102, and previous locations of the stabilization machine 102. The metering information may include the volume delivered and the rate of delivery of dry additive 136 and/or fluid 138 by the first stabilization machine 102a.

Block 710 of the method 700 disclosed herein includes determining, by the first stabilization controller 160a based on the location data, the location of the worked surface area 154 (in this embodiment, that surface worked by the first stabilization machine 102a, a first rotary mixer 114a). The current and previous locations of the first stabilization machine 102a may be utilized by the first stabilization controller 160a to determine the geographic location and boundaries of the worked surface area 154 (FIG. 2).

Block 720 includes determining, by the first stabilization controller 160a based on the location data, the quantity of passes over the worked surface area 154. The current and previous locations of the first stabilization machine 102a may be utilized by the first stabilization controller 160a to determine the number of passes made by the first stabilization machine 102a over the worked surface area 154.

Block 730 includes determining, by the first stabilization controller 160a, the mixing depth 147 of the worked surface area 154 or portions of the worked surface area 154.

Block 740 includes determining, by the first stabilization controller 160a, the moisture content of the base material 128 (after mixing by the first stabilization machine 102a) of the worked surface area 154 or portions of the worked surface area 154.

Block 750 of the method 700 includes transmitting, by the first stabilization controller 160a to either the at least one compactor controller 168 or the at least one second stabilization controller 160b (or to both compactor controller(s) 168 and second stabilization controller(s) 160b), the location and/or boundaries of the worked surface area 154, the quantity of passes made by the first stabilization machine 102a over the worked surface area 154 or portions of the worked surface area 154, the mixing depth 147 of the worked surface area 154 or portions of the worked surface area 154, and the moisture content of the worked surface area 154 or portions of the worked surface area 154.

Block 760 of the method 700 includes displaying, on the respective compactor user interface 170 or the respective stabilization user interface 162 (of the second stabilization machine 102b) or both, the location and/or boundaries of the worked surface area 154, the quantity of passes made by the first stabilization machine 102a over the worked surface area 154 or portions of the worked surface area 154, the mixing depth 147 of the worked surface area 154 or portions of the worked surface area 154, and the moisture content of the worked surface area 154 or portions of the worked surface area 154. The information to be displayed may be received by the compactor user interface 170 from the compactor controller 168, and may be received by the second stabilization user interface 162 by the second stabilization controller 160b.

What is claimed is:

1. A system comprising:
a first stabilization machine including a first stabilization controller, the first stabilization controller configured to:
determine a resupply time for a dry additive or fluid that is applied by the first stabilization machine; and
transmit to a supply truck controller the resupply time and a current location of the first stabilization machine; and
a supply truck configured to transport the dry additive or the fluid, the supply truck including a supply truck user interface and the supply truck controller, the supply truck controller in communication with the first stabilization controller and the supply truck user interface, the supply truck controller configured to display on the supply truck user interface the resupply time and the location.

2. The system of claim 1 further comprising a compactor, the compactor including a compactor user interface and a compactor controller, the compactor controller in communication with the compactor user interface and the first stabilization controller, in which the first stabilization controller is further configured to determine a location of a worked surface area that has been worked by the first stabilization machine and to transmit to the compactor controller the location of the worked surface area, the compactor controller configured to display on the compactor user interface the location of the worked surface area.

3. The system of claim 2, in which the first stabilization controller is further configured to determine a quantity of passes made in a portion of the worked surface area by the first stabilization machine.

4. The system of claim 2, in which the first stabilization controller is further configured to determine a moisture content of the worked surface area, and to transmit the moisture content to the compactor controller.

5. The system of claim 1 further comprising a second stabilization machine, the second stabilization machine including a second stabilization user interface and a second stabilization controller, the second stabilization controller in communication with the second stabilization user interface and the first stabilization controller, the first stabilization controller further configured to determine a location of a worked surface area that has been worked by the first stabilization machine and to transmit to the second stabilization controller the location of the worked surface area, the second stabilization controller configured to display on the second stabilization user interface the location of the worked surface area.

6. The system of claim 5, in which the first stabilization controller is further configured to determine a mixing depth of the worked surface area, and to transmit the mixing depth to the second stabilization controller.

7. The system of claim 5, in which the first stabilization controller is further configured to determine a moisture content of the worked surface area, and to transmit the moisture content to the second stabilization controller.

8. The system of claim 5, wherein the first stabilization machine is a first rotary mixer and the second stabilization machine is a second rotary mixer.

9. The system of claim 1 further including a site controller and a site user interface, the site controller and the site user interface remote from the first stabilization machine and the supply truck, in which the first stabilization controller is further configured to transmit to the site controller the resupply time and the current location, the site controller configured to display on the site user interface the resupply time and the current location.

10. A method of coordinating activities of a first rotary mixer in a system, the system including the first rotary mixer and a supply truck, the first rotary mixer including a first stabilization controller, the supply truck including a supply truck controller and a supply truck user interface, the supply truck controller in communication with the first stabilization controller and the supply truck user interface, the method comprising:
determining, by the first stabilization controller, a resupply time for a dry additive or a fluid that is applied by the first rotary mixer;
transmitting, by the first stabilization controller, the resupply time and a current location of the first rotary mixer to the supply truck controller; and
displaying the resupply time and the current location on the supply truck user interface.

11. The method of claim 10, further comprising determining, by the first stabilization controller, a location of a worked surface area that has been worked by the first rotary mixer.

12. The method of claim 11, further comprising transmitting to a compactor controller the location of the worked surface area, the compactor controller disposed on a compactor.

13. The method of claim 12, further comprising determining, by the first stabilization controller, a quantity of passes made by the first rotary mixer in a portion of the worked surface area and transmitting the quantity to the compactor controller.

14. The method of claim 13, further comprising determining, by the first stabilization controller, a mixing depth or a moisture content of the worked surface area, and transmitting the mixing depth or the moisture content to the compactor controller.

15. The method of claim 11, further comprising transmitting to a second stabilization controller the location of the worked surface area, the second stabilization controller disposed on a second rotary mixer.

16. The method of claim 15, further comprising determining, by the first stabilization controller, a quantity of passes made by the first rotary mixer in a portion of the worked surface area and transmitting the quantity to the second stabilization controller.

17. The method of claim 15, further comprising determining, by the first stabilization controller, a moisture content or mixing depth of the worked surface area and transmitting the moisture content or mixing depth to the second stabilization controller.

18. The method of claim 10, further comprising transmitting, by the first stabilization controller, to a site controller the resupply time and the current location, and displaying on a site user interface the resupply time and the current location, wherein the site controller and site user interface are remote from the first rotary mixer and the supply truck.

19. A system comprising:
a first rotary mixer including a first stabilization controller, the first stabilization controller configured to:
determine for the first rotary mixer a resupply time for a dry additive or fluid that is applied by the first rotary mixer, a location of a worked surface area that has been worked by the first rotary mixer, a quantity of passes made in a portion of the worked surface area by the first rotary mixer, a mixing depth of the worked surface area and a moisture content of the worked surface area; and
transmit to a compactor controller and to a second stabilization controller the location of the worked surface area, the quantity of passes, the mixing depth, and the moisture content;
a compactor including a compactor user interface and the compactor controller, the compactor controller in communication with the compactor user interface and the first stabilization controller, the compactor controller configured to display on the compactor user interface the location of the worked surface area, the quantity of passes, the mixing depth and the moisture content; and
a second rotary mixer including a stabilization user interface and the second stabilization controller, the second stabilization controller in communication with the stabilization user interface and the first stabilization controller, the second stabilization controller configured to display on the stabilization user interface the location of the worked surface area, the quantity of passes, the mixing depth and the moisture content.

20. The system of claim 19, in which the first stabilization controller is further configured to transmit to a supply truck controller the resupply time and a current location of the first rotary mixer, the system further comprising a supply truck configured to transport the dry additive or fluid, the supply truck including a supply truck user interface and the supply truck controller, the supply truck controller in communication with the first stabilization controller and the supply truck user interface, the supply truck controller configured to display on the supply truck user interface the resupply time and the current location.
